SEQUENCE LISTING (14 sequences)

SEQ ID NO: 1 - DNA, 42 bp, Artificial Sequence
  ZFP-Fwd
gcctggtgccgcgcggcagcccgaaaaagaaacgcaaagtgc

SEQ ID NO: 2 - DNA, 42 bp, Artificial Sequence
  ZFP-SOD1-Rev
gctttggtggccatggatccaccggtatgtgttctttgatgg

SEQ ID NO: 3 - DNA, 81 bp, Artificial Sequence
  the nucleotide sequence encoding the zinc finger protein
gaaaaaccatacaaatgcccagaatgcggaaaatcttttagtgcctcagctgccctcgtcgcccatcaaagaacacatacc

SEQ ID NO: 4 - DNA, 9 bp, Artificial Sequence
  The nucleotide sequence encoding the linker peptide
ggtggatcc

SEQ ID NO: 5 - DNA, 462 bp, Artificial Sequence
  The nucleotide sequence encoding the superoxide dismutase
atggccaccaaagcggtctgcgttttaaaaggggatggcccggtgcaaggcattattaatttcgaacaaaaagagagcaatggtccggttaaagtgtggggtagtatcaaaggcctgaccgagggtctgcatggctttcatgtgcatgaatttggcgataacaccgctggttgcacgtcagccggcccgcactttaatcctctgtcccgtaagcacggcggcccgaaggatgaggagcgtcacgtcggcgatctgggtaatgttactgccgataaggatggggtggccgatgtttccattgaagattctgtcatctcattgagtggggaccactgtatcattgggcgtaccttagtggtccatgaaaaggcagacgacctgggtaagggcggaaatgaagaatccaccaaaacgggcaatgctggttcacgtttagcgtgtggtgtgattggtatcgcccaa

SEQ ID NO: 6 - DNA, 552 bp, Artificial Sequence
  The nucleotide sequence encoding the domain of the fusion protein
gaaaaaccatacaaatgcccagaatgcggaaaatcttttagtgcctcagctgccctcgtcgcccatcaaagaacacataccggtggatccatggccaccaaagcggtctgcgttttaaaaggggatggcccggtgcaaggcattattaatttcgaacaaaaagagagcaatggtccggttaaagtgtggggtagtatcaaaggcctgaccgagggtctgcatggctttcatgtgcatgaatttggcgataacaccgctggttgcacgtcagccggcccgcactttaatcctctgtcccgtaagcacggcggcccgaaggatgaggagcgtcacgtcggcgatctgggtaatgttactgccgataaggatggggtggccgatgtttccattgaagattctgtcatctcattgagtggggaccactgtatcattgggcgtaccttagtggtccatgaaaaggcagacgacctgggtaagggcggaaatgaagaatccaccaaaacgggcaatgctggttcacgtttagcgtgtggtgtgattggtatcgcccaa

SEQ ID NO: 7 - DNA, 18 bp, Artificial Sequence
  The nucleotide sequence encoding the His tag
catcatcatcatcatcac

SEQ ID NO: 8 - DNA, 642 bp, Artificial Sequence
  The nucleotide sequence encoding the fusion protein with the tag
atgggcagcagccatcatcatcatcatcacagcagcggcctggtgccgcgcggcagcccgaaaaagaaacgcaaagtgctcgagcccggggaaaaaccatacaaatgcccagaatgcggaaaatcttttagtgcctcagctgccctcgtcgcccatcaaagaacacataccggtggatccatggccaccaaagcggtctgcgttttaaaaggggatggcccggtgcaaggcattattaatttcgaacaaaaagagagcaatggtccggttaaagtgtggggtagtatcaaaggcctgaccgagggtctgcatggctttcatgtgcatgaatttggcgataacaccgctggttgcacgtcagccggcccgcactttaatcctctgtcccgtaagcacggcggcccgaaggatgaggagcgtcacgtcggcgatctgggtaatgttactgccgataaggatggggtggccgatgtttccattgaagattctgtcatctcattgagtggggaccactgtatcattgggcgtaccttagtggtccatgaaaaggcagacgacctgggtaagggcggaaatgaagaatccaccaaaacgggcaatgctggttcacgtttagcgtgtggtgtgattggtatcgcccaa

SEQ ID NO: 9 - protein, 27 aa, Artificial Sequence
  the amino acid sequence of the zinc finger protein
EKPYKCPECGKSFSASAALVAHQRTHT

SEQ ID NO: 10 - protein, 3 aa, Artificial Sequence
  The amino acid sequence of the linker peptide
GGS

SEQ ID NO: 11 - protein, 154 aa, Artificial Sequence [sequence omitted]

SEQ ID NO: 12 - protein, 184 aa, Artificial Sequence
  the amino acid sequence of the domain of said fusion protein
EKPYKCPECGKSFSASAALVAHQRTHTGGSMATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 13 - protein, 6 aa, Artificial Sequence
  The amino acid sequence of the His tag
HHHHHH

SEQ ID NO: 14 - protein, 214 aa, Artificial Sequence
  The amino acid sequence of the ZFP-SOD1 protein with the His tag
MGSSHHHHHHSSGLVPRGSPKKKRKVLEPGEKPYKCPECGKSFSASAALVAHQRTHTGGSMATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

(19) United States
(12) Patent Application Publication
(10) Pub. No.: US 2022/0135629 A1
(43) Pub. Date: May 5, 2022

(54) Zinc Finger Protein-Superoxide Dismutase Fusion Protein With Cell Membrane Penetrating Property

(71) Applicant: SHANGHAITECH UNIVERSITY, Shanghai (CN)

(72) Inventors: Jia LIU, Shanghai (CN); Biao JIANG, Shanghai (CN); Peixiang MA, Shanghai (CN)

(21) Appl. No.: 17/085,157

(22) Filed: Oct. 30, 2020

(51) Int. Cl. C07K 14/47; C07K 14/52

(52) U.S. Cl. CPC C07K 14/47; C07K 14/52; A61K 38/00; C07K 2319/81; C07K 2319/71

(57) ABSTRACT

The present disclosure belongs to the field of biotechnology, and particularly relates to a zinc finger protein-superoxide dismutase fusion protein with a cell membrane penetrating property, and a preparation and an application thereof. A domain of the fusion protein described in the present disclosure comprises a zinc finger protein and superoxide dismutase, and the fusion protein can enter cells to exert an antioxidant activity.

### REFERENCE TO A SEQUENCE LISTING

This application contains references to amino acid sequences and/or nucleic acid sequences which have been submitted concurrently herewith as the sequence listing text file entitled “Sequence Listing.TXT,” file size 9,986 bytes, created on Feb. 18, 2021. The aforementioned sequence listing is hereby incorporated by reference in its entirety pursuant to 37 C.F.R. § 1.52(e)(5).

### TECHNICAL FIELD

The present disclosure belongs to the field of biotechnology, and particularly relates to a zinc finger protein-superoxide dismutase fusion protein with a cell membrane penetrating property, and a preparation and an application thereof.

### PRIOR ARTS

The selective permeability of cellular membranes separates the cellular components from exogenous molecules. This proves a major challenge for the delivery of therapeutic agents into cells. Various bioactive agents including genes, proteins and virus can be efficiently internalized into cells. Among these agents, the direct delivery of functional proteins holds great promise for therapeutic applications because of its safety and efficiency. Protein delivery does not depend on the transcription and translation of imported nucleic acids. Therefore, the delivered proteins can action rapidly and then be degraded by proteasome system, leading to less risk of mutagenesis. One major obstacle of protein delivery is the selectivity of cell membrane. Numerous membrane perturbation techniques, such as microinjection and electroporation, have been investigated for accelerating protein delivery. However, these membrane disruption technologies are often associated with low efficiency, high toxicity, penurious bioavailability, and poor specificity. In addition to the physical membrane puncture methods, many biochemical agents were developed to facilitate protein delivery, such as supercharged transduction domains, nanoparticles, liposomes, virus-like particles and polymeric microsphere. In the preclinical or clinical practice, these strategies can be associated with drawbacks such as inefficient cellular uptake, poor stability, inadvertent cell-type specificity, low rate of endosomal escape or toxicity. In the late 1980s, a naturally occurring peptide from TAT trans-activating factor of human immunodeficiency virus (HIV) was found to possess inherent cell-penetrating ability. In the following years, a series of natural peptides with similar cell permeability were identified, which were later recognized as cell-penetrating peptides (CPPs). Based on the features of naturally occurring CPPs, artificial or chimeric CPPs were designed. CPPs often have minimal cytotoxicity and can be applied to various cell types for the delivery of a wide range of cargo molecules with different molecular weights. These CPPs can be either genetically fused to or chemically conjugated to the cargo proteins.

### CONTENT OF THE PRESENT INVENTION

In order to overcome the problems in the prior art, an object of the present disclosure is to provide a zinc finger protein-superoxide dismutase fusion protein with a cell membrane penetrating property, and a preparation and an application thereof.

In order to achieve the above-mentioned object and other related objects, the present disclosure uses the following technical solutions:

in a first aspect of the present disclosure, provided is a fusion protein, wherein a domain thereof comprises a zinc finger protein and superoxide dismutase.

Further, the amino acid sequence of the zinc finger protein is as shown in SEQ ID NO. 9, specifically is:

EKPYKCPECGKSFSASAALVAHQRTHT.

Further, the amino acid sequence of the superoxide dismutase is as shown in

SEQ ID NO. 11, specifically is:

MATKAVCVLKGDGPVQGIINFEQKESNGPVKVWGS
IKGLTEGLHGFHVHEFGDNTAGCTSAGPHFNPLSR
KHGGPKDEERHVGDLGNVTADKDGVADVSIEDSVI
SLSGDHCIIGRTLVVHEKADDLGKGGNEESTKTGN
AGSRLACGVIGIAQ.

Further, the zinc finger protein and superoxide dismutase are connected by a linker peptide.

Further, the number of amino acids of the linker peptide is ≥2, and the linker peptide consists of glycine (Gly) and serine (Ser), and is obtained by using a multi-unit connection in which G2S (i.e. an amino acid sequence of GGS) is used as a unit.

Further, the amino acid sequence of the linker peptide is as shown in SEQ ID NO. 10, specifically is: GGS.

The present disclosure has no special requirements for the order of connection, as long as it does not limit the object of the present disclosure. For example, the C-terminus of the zinc finger protein can be connected to the N-terminus of the superoxide dismutase; or the C-terminus of the superoxide dismutase can be connected to the N-terminus of the zinc finger protein.

That is, the general formula of the domain of the fusion protein is: zinc finger protein-linker peptide-superoxide dismutase or superoxide dismutase-linker peptide-zinc finger protein.

Preferably, the domain of said fusion protein, from the N-terminus to the C-terminus, successively comprises the zinc finger protein, linker peptide, and superoxide dismutase.

Further, in a preferred embodiment of the present disclosure, it has listed that the amino acid sequence of the domain of said fusion protein is as shown in SEQ ID NO. 12, specifically is:

EKPYKCPECGKSFSASAALVAHQRTHTGGSMATKA
VCVLKGDGPVQGIINFEQKESNGPVKVWGSIKGLT
EGLHGFHVHEFGDNTAGCTSAGPHFNPLSRKHGGP
KDEERHVGDLGNVTADKDGVADVSIEDSVISLSGD
HCIIGRTLVVHEKADDLGKGGNEESTKTGNAGSRL
ACGVIGIAQ.

However, it is not limited by the specific form listed in the preferred embodiment of the present disclosure.

Further, the fusion protein can comprise a tag. The tag is used for purifying a protein.

For example, the tag can be an affinity purification tag, such as His tag, MBP tag, GST tag, and FLAG tag.

The tag can be connected to the N-terminus or C-terminus of the domain of the fusion protein, as long as it does not affect the functions of the tag and the domain of the fusion protein.

In a preferred embodiment of the present disclosure, it has listed that the amino acid sequence of the fusion protein with the tag is as shown in SEQ ID NO. 14, specifically is:

MGSSHEIHHHHSSGLVPRGSPKKKRKVLEPGEKPY
KCPECGKSFSASAALVAHQRTHTGGSMATKAVCVL
KGDGPVQGIINFEQKESNGPVKVWGSIKGLTEGLH
GFHVHEFGDNTAGCTSAGPHFNPLSRKHGGPKDEE
RHVGDLGNVTADKDGVADVSIEDSVISLSGDHCII
GRTLVVHEKADDLGKGGNEESTKTGNAGSRLACGV
IGIAQ.

However, it is not limited by the specific form listed in the preferred embodiment of the present disclosure.

In a second aspect of the present disclosure, provided is an isolated polynucleotide (i.e. a DNA molecule) encoding the above-mentioned fusion protein.

The polynucleotide encoding the fusion protein of the present disclosure can be in the forms of DNA or RNA. The DNA form comprises cDNA, genomic DNA or artificially synthetic DNA. DNA can be single stranded or double stranded.

The polynucleotide encoding the fusion protein of the present disclosure can be prepared by any appropriate technique well known to those skilled in the art. The technique can be found in the general description in the art, such as Molecular Cloning: A Laboratory Manual (J. Sambrook et al., Science Press, 1995). The technique comprises, but not limited to, methods such as recombinant DNA technology and chemical synthesis; for example, using the overlap extension PCR method.

For example, the nucleotide sequence encoding the zinc finger protein is as shown in SEQ ID NO. 3, specifically is:

gaaaaaccatacaaatgcccagaatgcggaaaatc
ttttagtgcctcagctgccctcgtcgcccatcaaa
gaacacatacc.

The nucleotide sequence encoding the superoxide dismutase is as shown in SEQ ID NO. 5, specifically is:

atggccaccaaagcggtctgcgttttaaaaggggat
ggcccggtgcaaggcattattaatttcgaacaaaa
agagagcaatggtccggttaaagtgtggggtagta
tcaaaggcctgaccgagggtctgcatggctttcat
gtgcatgaatttggcgataacaccgctggttgcac
gtcagccggcccgcactttaatcctctgtcccgta
agcacggcggcccgaaggatgaggagcgtcacgtc
ggcgatctgggtaatgttactgccgataaggatgg
ggtggccgatgrnccattgaagattctgtcatctc
attgagtggggaccactgtatcattgggcgtacct
tagtggtccatgaaaaggcagacgacctgggtaag
ggcggaaatgaagaatccaccaaaacgggcaatgc
tggttcacgtttagcgtgtggtgtgattggtatcg
cccaa.

The nucleotide sequence encoding the linker peptide is as shown in SEQ ID NO. 4, specifically is: ggtggatcc.

The nucleotide sequence encoding the domain of the fusion protein is as shown in SEQ ID NO. 6, specifically is:

gaaaaaccatacaaatgcccagaatgcggaaaatc
ttttagtgcctcagctgccctcgtcgcccatcaaa
gaacacataccggtggatccatggccaccaaagcg
gtctgcgttttaaaaggggatggcccggtgcaagg
cattattaatttcgaacaaaaagagagcaatggtc
cggttaaagtgtggggtagtatcaaaggcctgacc
gagggtctgcatggctttcatgtgcatgaatttgg
cgataacaccgctggttgcacgtcagccggcccgc
actttaatcctctgtcccgtaagcacggcggcccg
aaggatgaggagcgtcacgtcggcgatctgggtaa
tgttactgccgataaggatggggtggccgatgttt
ccattgaagattctgtcatctcattgagtggggac
cactgtatcattgggcgtaccttagtggtccatga
aaaggcagacgacctgggtaagggcggaaatgaag
aatccaccaaaacgggcaatgctggttcacgttta
gcgtgtggtgtgattggtatcgcccaa.

The nucleotide sequence encoding the fusion protein with the tag is as shown in SEQ ID NO. 8, specifically is:

Atgggcagcagccatcatcatcatcatcacagcag
cggcctggtgccgcgcggcagcccgaaaaagaaac
gcaaagtgctcgagcccggggaaaaaccatacaaa
tgcccagaatgcggaaaatcttttagtgcctcagc
tgccctcgtcgcccatcaaagaacacataccggtg
gatccatggccaccaaagcggtctgcgttttaaaa
ggggatggcccggtgcaaggcattattaatttcga
acaaaaagagagcaatggtccggttaaagtgtggg
gtagtatcaaaggcctgaccgagggtctgcatggc
tttcatgtgcatgaatttggcgataacaccgctgg
ttgcacgtcagccggcccgcactttaatcctctgt
cccgtaagcacggcggcccgaaggatgaggagcgt
cacgtcggcgatctgggtaatgttactgccgataa
ggatggggtggccgatgtttccattgaagattctg
tcatctcattgagtggggaccactgtatcattggg
cgtaccttagtggtccatgaaaaggcagacgacct
gggtaagggcggaaatgaagaatccaccaaaacgg
gcaatgctggttcacgtttagcgtgtggtgtgatt
ggtatcgcccaa.

In a third aspect of the present disclosure, provided is a recombinant expression vector comprising the above-mentioned isolated polynucleotide.

The recombinant expression vector of the present disclosure comprises the polynucleotide encoding the fusion protein. Methods well known to those skilled in the art can be used for constructing the expression vector. These methods comprise recombinant DNA technology, DNA synthetic technology, etc. The DNA encoding the fusion protein can be effectively connected to a multiple cloning site in the vector for guiding mRNA synthesis and then expressing the protein, or for homologous recombination. In a preferred embodiment of the present disclosure, the expression vector can use pET28, pMAL, pcDNA, etc.

In a fourth aspect of the present disclosure, provided is a host cell comprising the above-mentioned recombinant expression vector or the exogenous, integrated in the genome of the host cell, above-mentioned isolated polynucleotide.

In a preferred embodiment of the present disclosure, the host cell can use BL21 Escherichia coli, an insect cell, a CHO cell, an HEK293 cell, etc.

In a fifth aspect of the present disclosure, provided is a method for preparing the above-mentioned fusion protein, comprising the following steps:

(1) constructing a recombinant expression vector comprising the polynucleotide encoding the fusion protein, then transforming the recombinant expression vector into a host cell to induce expression, and isolating and obtaining the fusion protein from an expression product;

or

(2) culturing the above-mentioned host cell under suitable conditions, such that the host cell expresses the fusion protein, and then isolating and purifying same to obtain the fusion protein.

In a preferred embodiment of the present disclosure, the expression vector can use pET28, pMAL, and pcDNA.

The host cell can use BL21 Escherichia coli, an insect cell, a CHO cell, an HEK293 cell, etc.

In a sixth aspect of the present disclosure, provided is the use of the above-mentioned fusion protein, isolated polynucleotide, recombinant expression vector, or host cell in preparing antioxidant-related products.

Preferably, the antioxidant products are selected from cerebral ischemia therapeutic products, cancer therapeutic products, AIDS therapeutic products, amyotrophic lateral sclerosis therapeutic products, anti-inflammatory response products, Parkinson syndrome therapeutic products, and facial cosmetic products.

Compared with the prior art, the present disclosure has the following beneficial effects:

(1) the fusion protein of the present disclosure is easier to express and purify than an SOD protein fused to a traditional cell membrane penetrating peptide;

(2) the fusion protein of the present disclosure has a lower risk of causing an immune response; the zinc finger protein and SOD are both naturally occurring proteins in the human body; and

(3) the fusion protein of the present disclosure can present an active SOD enzyme into a cell, but traditional methods often inactivate the enzyme.

### BRIEF DESCRIPTION OF THE DRAWINGS

FIG. 1: ZFP-SOD1 protein purification. (a) Schematic presentation of ZFP-hSOD1 construct. (b) SDS-PAGE of purified ZFP-hSOD1 protein. Arrow indicates the target protein band.

FIG. 2: Internalized SOD1 protein in Hela cells, as determined by SOD activity. n.s., not significant. *, P<0.05.

### DETAILED DESCRIPTION OF THE PREFERRED EMBODIMENT

The inventors of the present application identified the Cys₂-His₂ zinc-finger proteins (ZFPs) as a novel protein delivery system. ZFPs are inherently cell-permeable due to the constellation of six positively charged residues on the protein surface. We eliminated the DNA binding ability of ZFPs by mutating the residues responsible for DNA-binding in the a-helices. The engineered zinc-finger proteins (ZFPs) retained cell-permeability and can be used as a fusion tag to deliver cargo proteins. The cellular uptake efficacy is tunable by adjusting the number of tandem ZFP domains, which increases the plasticity for different applications. ZFP domains can mediate the efficient intracellular delivery of protein cargos such as green fluorescent protein (GFP) and Fok I nuclease. In addition to transformed cell lines, ZFPs can facilitate the delivery of cargo proteins into primary cells and stem cells, which is important for the therapeutic applications.

Superoxide dismutase (SOD) family include a group of well-studied antioxidant enzymes, i.e. SOD1, SOD2 and SOD3. SODs play a fundamental role in attenuating oxidative stress from cellular reactive oxygen species (ROS). The disorder of ROS can contribute to the occurrence and progression of a variety of diseases. Preclinical and clinical studies have shown great therapeutic potential of SODs. SODs have been employed for a wide range of medical indications such as ischemia reperfusion injury, transplant induced reperfusion injury, inflammation, Parkinson's disease, cancer and acquired immune deficiency syndrome (AIDS).

The present application provides the construction, expression, purification and cell activity assays of ZFP-SOD1 fusion protein for the first time, and proves that the ZFP-SOD1 can penetrate cell membranes and exert SOD1 antioxidant activity inside cells.

Before further describing the specific embodiments of the present disclosure, it is to be understood that the scope of protection of the present disclosure is not limited to the following specific particular embodiments; it is also to be understood that the terms used in the embodiments of the present disclosure are used for describing the specific particular embodiments, rather than limiting the scope of protection of the present disclosure. In the following embodiments, experimental methods without specifying specific conditions are generally carried out according to conventional conditions or conditions suggested by each manufacturer.

When numerical ranges are given in the embodiments, it is to be understood that the two endpoints of each numerical range and any value between the two endpoints can be selected, unless specified otherwise in the present disclosure. Unless defined otherwise, all technical and scientific terms used in the present disclosure have the same meanings commonly understood by those of skill in the art. In addition to the specific methods, devices, and materials used in the embodiments, according to the knowledge in the prior art and the description of the present disclosure, those of skill in the art can also use any prior art methods, devices, and materials which are similar or equal to the methods, devices, and materials described in the embodiments of the present disclosure to realize the present disclosure.

Unless specified otherwise, the experimental methods, detection methods, and preparation methods disclosed in the present disclosure all use conventional molecular biological, biochemical, chromatin structure and analysis, analytical chemical, cell culture, and recombinant DNA technology in the art, and other conventional technology in related fields. The technology has been completely described in existing documents. For details, reference can be made to: Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; METHODS IN MOLECULAR BIOLOGY, Vol. 119, Chromatin Protocols (P.B. Becker, ed) Humana Press, Totowa, 1999, etc.

### EXAMPLE 1

I. Materials

1. Construction of Expression Plasmids

1) pET28a plasmid encoding human SOD1 gene optimized for Escherichia coli expression (available from commercial gene synthesis service suppliers).

2) ZFP containing plasmid.

3) DNA polymerase.

4) Deoxynucleotide mixture, including dATP, dCTP, dGTP and dTTP.

5) PCR reaction buffers.

6) Sterile water.

7) DNA staining reagents.

8) Homologous recombination enzymes.

9) DH5α E. coli competent cells.

10) Lysogeny broth (LB) medium.

11) Agar, bacteriological grade.

12) Plasmid DNA extraction kit.

13) Gradient thermal cycler for PCR.

14) Agarose gel electrophoresis reagents and equipment.

15) UV trans-illuminator.

16) Centrifuge.

17) Water bath.

2. Protein Expression and Purification

1) Plasmids encoding recombinant ZFP-SOD1 proteins.

2) BL21(DE3) competent E. coli cells.

3) Agar, bacteriological grade.

4) 50 mg/mL Kanamycin stock solution

5) 1 M IPTG (isopropil-β-D-1-tiogalattopiranoside).

6) Ni-NTA Agarose.

7) 1M Tris-HCl pH 8.0.

8) 4 M Imidazole stock solution.

9) 100 mM ZnCl₂ stock solution.

10) 100 mM MgCl₂ stock solution.

11) Phenylmethylsulfonyl fuoride (PMSF) (100 mM in ethanol).

12) Lysis buffer: 50 mM Tris-HCl, pH 8.0, 500 mM NaCl, 100 □M ZnCl2, 1 mM MgCl2, 1 mM PMSF and 5 mM imidazole.

13) Wash buffer: 50 mM Tris-HCl, pH 8.0, 500 mM NaCl, 100 □M ZnCl2, 1 mM MgCl2 and 30 mM imidazole.

14) Elution buffer: 50 mM Tris-HCl, pH 8.0, 500 mM NaCl, 100 □M ZnCl2, 1 mM MgCl2 and 300 mM imidazole.

15) Storage buffer: 50 mM Tris-HCl, pH 8.0, 500 mM NaCl, 100 □M ZnCl2, 1 mM MgCl2 and 10% glycerol.

16) Baffled cell culture flasks.

17) Concentrator.

18) 4-20% Tris-Glycine SDS-PAGE.

19) SDS protein loading dye.

20) BCA protein assay kit.

21) Liquid nitrogen.

3. Protein Transduction

1) Purified ZFP-SOD1 proteins.

2) Class II biosafety cabinet.

3) Cell incubator.

4) Bright field phase contrast microscope.

5) Dulbecco's modified eagle's medium (DMEM).

6) Fetal bovine serum (FBS).

7) Penicillin and streptomycin solution.

8) Phosphate-buffered saline (PBS).

9) 9 mM ZnCl2.

10) Triton X-100

11) 0.05% trypsin-EDTA solution with phenol red.

12) Hela cells.

13) Tissue culture flasks.

14) 24-well flat bottom tissue culture plates.

15) Centrifuge.

16) SOD assay kit.

II. Methods

1. Construction of ZFP-SOD1 (zinc finger protein-superoxide dismutase fusion protein) Expression Plasmids

1) Miniprep ZFP-encoding plasmid (pET28-ZiF1-EmGFP) and synthesize human SOD genes (superoxide dismutase genes) in pET28a vector (pET28a-hSOD1) using a commercial gene synthesis service.

2) PCR amplify ZFP genes (zinc finger protein genes)

from the plasmid pET-1F-ZiF with the primers

ZFP-Fwd
(5′-GCCTGGTGCCGCGCGGCAGCCCG
AAAAAGAAACGCAAAGTGC-3′)
and
ZFP-SOD1-Rev
(5′-GCTTTGGTGGCCATGGATCCACC
GGTATGTGTTCTTTGATGG-3′).

3) Prepare PCR mixture to amplify the genes encoding ZFP domain: use 5 ng of template DNA, 5 μL of 10× polymerase buffer, 1 Units (U) of Taq DNA polymerase, 0.2 mM dNTP mixture and 0.2 μM of each primer in a 50 μL solution. PCR conditions are cycled using the following settings: 95° C. for 5 min, 30 cycles of 95° C. for 30 sec, 58° C. for 30 sec and 72° C. for 1 min and final extension at 72° C. for 10 min. Purify the PCR product by gel extraction and determine DNA concentration using a spectrophotometer measuring Abs260×50 ng/μL.

4) Digest the 1 μg of pET28a-hSOD1 plasmid with 10 U of each Ndel and BamHI in recommended buffer for 3 h at 37° C. Visualize DNA by agarose gel electrophoresis using a DNA staining dye, such as gel red.

5) Purify the digested plasmid by gel extraction kit and determine DNA concentration by a spectrophotometer measuring Abs260×50 ng/μL.

6) Perform homologous recombination reaction for constructing ZFP-SOD1 fusion protein as follows: 0.06 pmol ZFP PCR product, 0.03 pmol linearized pET28a-hSOD1 plasmid DNA, 2 μL recombination enzyme such as Exnase II, 4 μL 5× recombination buffer and deionized water up to 20 μL. Incubate the mixture at 37° C. for 30 min.

7) Thaw 200 μL of chemically competent DH5α E.coli cells on ice, mix gently with 20 μL of recombination products and then incubate on ice for 30 min.

8) Heat shock the mixture at 42° C. for 45-90 s and recover the cells in 900 μL LB medium for 1 h at 37° C. with shaking.

9) Spread 100 μL of recovery culture on a LB agar plate supplemented with 50·g/mL kanamycin and incubate overnight at 37° C.

10) The following day, inoculate a single colony into 5 mL of LB culture containing 50 μg/mL kanamycin and culture overnight at 37° C.

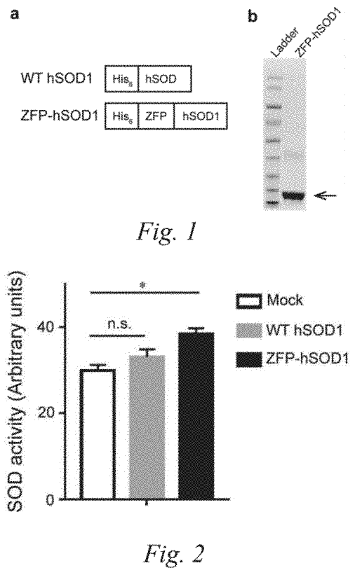

11) Miniprep pET28a-ZFP-SOD1 plasmid and confirm the construct (schematic presentation of ZFP-hSOD1 construct is shown in FIG. 1) by DNA sequencing using a primer binding to the T7 promoter (5′-TAATAC GAC T CAC TATAGGG-3′).

As can be seen from the results: the domain of the ZFP-SOD1 protein (zinc finger protein-superoxide dismutase fusion protein), from the N-terminus to the C-terminus, successively comprises the zinc finger protein, linker peptide, and superoxide dismutase, and full-length genes of the domain of the fusion protein all have correct sequences, and are all in line with expectations.

That is, in the domain of the fusion protein, the nucleotide sequence encoding the zinc finger protein is as shown in SEQ ID NO. 3, specifically is: gaaaaaccatacaaatgcccagaatgeggaaaatcttttagtgcctcagctgccctcgtcgcccatcaaagaacacatacc. The nucleotide sequence encoding the linker peptide is as shown in SEQ ID NO. 4, specifically is: ggtggatcc. The nucleotide sequence encoding the superoxide dismutase is as shown in SEQ ID NO. 5, specifically is:

atggccaccaaagcggtctgcgttttaaaagggga
tggcccggtgcaaggcattattaatttcgaacaaa
aagagagcaatggtccggttaaagtgtggggtagt
atcaaaggcctgaccgagggtctgcatggctttca
tgtgcatgaatttggcgataacaccgctggttgca
cgtcagccggcccgcactttaatcctctgtcccgt
aagcacggcggcccgaaggatgaggagcgtcacgt
cggcgatctgggtaatgttactgccgataaggatg
gggtggccgatgtttccattgaagattctgtcatc
tcattgagtggggaccactgtatcattgggcgtac
cttagtggtccatgaaaaggcagacgacctgggta
agggcggaaatgaagaatccaccaaaacgggcaat
gctggttcacgtttagcgtgtggtgtgattggtat
cgcccaa.

The nucleotide sequence encoding the domain of the ZFP-SOD1 protein (zinc finger protein-superoxide dismutase fusion protein) is as shown in SEQ ID NO. 6, specifically is:

gaaaaaccatacaaatgcccagaatgcggaaaatc
ttttagtgcctcagctgccctcgtcgcccatcaaa
gaacacataccggtggatccatggccaccaaagcg
gtctgcgttttaaaaggggatggcccggtgcaagg
cattattaatttcgaacaaaaagagagcaatggtc
cggttaaagtgtggggtagtatcaaaggcctgacc
gagggtctgcatggctttcatgtgcatgaatttgg
cgataacaccgctggttgcacgtcagccggcccgc
actttaatcctctgtcccgtaagcacggcggcccg
aaggatgaggagcgtcacgtcggcgatctgggtaa
tgttactgccgataaggatggggtggccgatgttt
ccattgaagattctgtcatctcattgagtggggac
cactgtatcattgggcgtaccttagtggtccatga
aaaggcagacgacctgggtaagggcggaaatgaag
aatccaccaaaacgggcaatgctggttcacgttta
gcgtgtggtgtgattggtatcgcccaa.

In order to purify the protein conveniently, an His tag is added to the N-terminus of the domain of the ZFP-SOD1 protein (zinc finger protein-superoxide dismutase fusion protein).

The nucleotide sequence encoding the His tag is as shown in SEQ ID NO. 7, specifically is: catcatcatcatcatcac.

The nucleotide sequence encoding the ZFP-SOD1 protein with the His tag is as shown in SEQ ID NO. 8, specifically is:

atgggcagcagccatcatcatcatcatcacagcag
cggcctggtgccgcgcggcagcccgaaaaagaaac
gcaaagtgctcgagcccggggaaaaaccatacaaa
tgcccagaatgcggaaaatcttttagtgcctcagc
tgccctcgtcgcccatcaaagaacacataccggtg
gatccatggccaccaaagcggtctgcgttttaaaa
ggggatggcccggtgcaaggcattattaatttcga
acaaaaagagagcaatggtccggttaaagtgtggg
gtagtatcaaaggcctgaccgagggtctgcatggc
tttcatgtgcatgaatttggcgataacaccgctgg
ttgcacgtcagccggcccgcactttaatcctctgt
cccgtaagcacggcggcccgaaggatgaggagcgt
cacgtcggcgatctgggtaatgttactgccgataa
ggatggggtggccgatgtttccattgaagattctg
tcatctcattgagtggggaccactgtatcattggg
cgtaccttagtggtccatgaaaaggcagacgacct
gggtaagggcggaaatgaagaatccaccaaaacgg
gcaatgctggttcacgtttagcgtgtggtgtgatt
ggtatcgcccaa.

2. Expression and purification of ZFP-SOD1 protein (zinc finger protein-superoxide dismutase fusion protein)

1) Thaw 50 μL of chemically competent BL21 E. coli cells on ice and mix gently with 100 ng of pET28a-ZFP-SOD1 plasmid. Perform heat shock transformation as described in steps 7)-9) of part II.1.

2) The following day, inoculate a single colony into 10 mL of LB medium containing 50 μg/mL kanamycin and grow overnight at 37° C. with shaking.

3) The following day, dilute the 10 mL of overnight culture into 1 L of LB medium supplemented with 50 μg/mL kanamycin. Grow the culture at 37° C. with shaking to an optical density at 600 nm (OD600) of 0.6-0.8 and induce protein expression with 0.5 mM IPTG. After 6 h of expression at 37° C., harvest cells by centrifugation at 5,000×g for 20 min at 4° C.

4) Resuspend 1 g cell pellets in 5 mL of lysis buffer. Lyse the cells on ice with cell distributor or sonication.

5) Centrifuge the cell lysate at 40,000×g for 30 min at 4° C. and transfer the supernatant into a fresh collection tube. For optimum results, perform all the following steps at 4° C.

6) Flow the supernatant through a column pre-packed with 1 mL of equilibrated Ni-NTA agarose. Wash the resin with 20 mL of wash buffer.

7) Elute the protein with 5 mL of elution buffer.

8) Buffer exchange the eluted protein with storage buffer and concentrate the protein to at least 40 μM using a spin concentrator following manufacturer's instructions.

9) Determine protein concentration by BCA or Bradford assay.

10) Mix 2 μL of purified proteins with 2 μL 2×SDS-PAGE loading dye, boil at 95° C. for 10 min and then resolve on 4-20% Tris-Glycine SDS-PAGE to assess protein purity. The results are as shown in (b) of FIG. 1, and show that: the ZFP-SOD1 protein (zinc finger protein-superoxide dismutase fusion protein) can be successfully recombined and expressed in vitro, and a fusion protein with a relatively high purity can be obtained.

By means of N/C-terminal sequence analysis, the results show that the domain of the ZFP-SOD1 protein (zinc finger protein-superoxide dismutase fusion protein), from the N-terminus to the C-terminus, successively comprises the zinc finger protein, linker peptide, and superoxide dismutase, and the frames of the expressed fusion protein are all read correctly, and are consistent with the theoretical N/C-terminal amino acid sequences.

That is: in the domain of the fusion protein, the amino acid sequence of the zinc finger protein is as shown in SEQ ID NO. 9, specifically is EKPYKCPECGKSFSASAALVAHQRTHT. The amino acid sequence of the linker peptide is as shown in SEQ ID NO. 10, specifically is: GGS. The amino acid sequence of the superoxide dismutase is as shown in SEQ ID NO. 11, specifically is:

MATKAVCVLKGDGPVQGIINFEQKESNGPVKVWGS
IKGLTEGLHGFHVHEFGDNTAGCTSAGPHFNPLSR
KHGGPKDEERHVGDLGNVTADKDGVADVSIEDSVI
SLSGDHCIIGRTLVVHEKADDLGKGGNEESTKTGN
AGSRLACGVIGIAQ.

The amino acid sequence of the domain of the ZFP-SOD1 protein (zinc finger protein-superoxide dismutase fusion protein) is as shown in SEQ ID NO. 12, specifically is:

EKPYKCPECGKSFSASAALVAHQRTHTGGSMATKA
VCVLKGDGPVQGIINFEQKESNGPVKVWGSIKGLT
EGLHGFHVHEFGDNTAGCTSAGPHFNPLSRKHGGP
KDEERHVGDLGNVTADKDGVADVSIEDSVISLSGD
HCIIGRTLVVHEKADDLGKGGNEESTKTGNAGSRL
ACGVIGIAQ.

In order to purify the protein conveniently, a His tag is added to the N-terminus of the domain of the ZFP-SOD1 protein (zinc finger protein-superoxide dismutase fusion protein).

The amino acid sequence of the His tag is as shown in SEQ ID NO. 13, specifically is: HEIREIHH. The amino acid sequence of the ZFP-SOD1 protein with the His tag is as shown in SEQ ID NO. 14, specifically is:

MGSSHHHHHHSSGLVPRGSPKKKRKVLEPGEKPY
KCPECGKSFSASAALVAHQRTHTGGSMATKAVCVL
KGDGPVQGIINFEQKESNGPVKVWGSIKGLTEGLH
GFHVHEFGDNTAGCTSAGPHFNPLSRKHGGPKDEE
RHVGDLGNVTADKDGVADVSIEDSVISLSGDHCII
GRTLVVHEKADDLGKGGNEESTKTGNAGSRLACGV
IGIAQ.

11. The protein is concentrated, rapidly frozen in liquid nitrogen, and stored at −80° C. .

3. ZFP-SOD1 Protein (zinc finger protein-superoxide dismutase fusion protein) transduction

1) Maintain HeLa cells in DMEM medium supplemented with 10% (v/v) FBS, 100 U/mL penicillin and 100 U/mL streptomycin at 37° C. in a fully humidified atmosphere with 5% CO₂.

2) Pre-coat a 24-well plate with 500 μL of 50 μg,/mL of poly-lysine for 30 to 60 min at 25° C. Seed Hela cells onto pre-coated plates at a density of 2×10⁵ cells per well.

3) At 24 h after seeding, remove medium from each well and wash with 500 μL, of pre-warmed serum-free DMEM.

4) Add to each well 250 μL, of SFM containing 2 μM of ZFP-SOD1 proteins and 100 μM ZnCl₂. Incubate at 37° C. for 1 h.

5) Remove media from cells and wash three times with 500 μL of calcium- and magnesium-free PBS supplemented with 0.5 mg/mL of heparin.

6) Rinse cells with 0.05% trypsin-EDTA, remove trypsin solution and then incubate at 37° C. for 2 min.

7) Lyse cells using 250 μL of PBS containing 0.1% (v/v) Triton X-100.

8) Use SOD assay kit such as SOD Determination Kit (Sigma-Aldrich, St. Louis, Mo., USA) to determine internalized SOD proteins. The results are as shown in FIG. 2, and show that the ZFP-SOD1 protein (zinc finger protein-superoxide dismutase fusion protein) can enter cells to exert an antioxidant activity. The ZFP-SOD1 protein can penetrate the cell membrane and enter the cell, thereby improving the antioxidant activity of the cell itself (improved from 30 to 40, and improved by about ⅓).

### COMPARATIVE EXAMPLE

The document of Kwon et al. (reference: Transduction of Cu, Zn-superoxide dismutase mediated by an HIV-1 Tat protein basic domain into mammalian cells) demonstrates that the cell presentation of SOD mediated by a cell membrane penetrating peptide Tat can only act on denatured SOD, but not act on SOD under physiological conditions. However, our results demonstrate that the zinc finger protein can present SOD under physiological conditions to a cell.

The above description merely relates to preferred embodiments of the present disclosure, but is not intended to limit the present disclosure in any formal and substantial ways. It should be noted that for those of skill in the art, without departing from the method of the present disclosure, several improvements and supplements can be further made, and these improvements and supplements also should fall within the scope of protection of the present disclosure. For those of skill in the art, without departing from the spirit and scope of the present disclosure, the equivalent changes of a little alterations, modifications and evolutions made according to the technical content revealed above are all equivalent embodiments of the present disclosure; meanwhile, any equivalent changes of alterations, modifications and evolutions on the above-mentioned embodiments according to the substantial technology of the present disclosure all still fall within the scope of the technical solution of the present disclosure.

1. A fusion protein, wherein a domain of the fusion protein comprises a zinc finger protein and superoxide dismutase.

2. The fusion protein as defined in claim 1, wherein the zinc finger protein and superoxide dismutase are connected by a linker peptide, preferably, the domain of the fusion protein, from the N-terminus to the C-terminus, successively comprises the zinc finger protein, linker peptide, and superoxide dismutase.

3. The fusion protein as defined in claim 1, wherein the fusion protein further comprises a tag.

4. The fusion protein as defined in claim 1, wherein the fusion protein further comprises any one or two of the following features: (1) the amino acid sequence of the zinc finger protein is as shown in SEQ ID NO. 9; and (2) the amino acid sequence of the superoxide dismutase is as shown in SEQ ID NO. 11.

5. The fusion protein as defined in claim 1, wherein the amino acid sequence of the domain of the fusion protein is as shown in SEQ ID NO. 12.

6. An isolated polynucleotide encoding the fusion protein as defined in claim 1.

7. A recombinant expression vector comprising the isolated polynucleotide as defined in claim 6.

8. A host cell comprising the recombinant expression vector as defined in claim 7 or an exogenous, integrated in the genome of the host cell, isolated polynucleotide as defined in claim 6.

9. A method for preparing the fusion protein as defined in claim 1, comprising the following steps: (1) constructing a recombinant expression vector comprising the polynucleotide encoding the fusion protein, then transforming the recombinant expression vector into a host cell to induce expression, and isolating and obtaining the fusion protein from an expression product; or (2) culturing the host cell as defined in claim 8 under suitable conditions, such that the host cell expresses the fusion protein, and then isolating and purifying same to obtain the fusion protein.

10. Use of the fusion protein as defined in claim 1 in preparing antioxidant-related products, preferably, the antioxidant products are selected from cerebral ischemia therapeutic products, cancer therapeutic products, AIDS therapeutic products, amyotrophic lateral sclerosis therapeutic products, anti-inflammatory response products, Parkinson syndrome therapeutic products, and facial cosmetic products.
